(19) United States
(12) Patent Application Publication
(10) Pub. No.: US 2012/0009125 A1
(43) Pub. Date: Jan. 12, 2012

(54) APOE4 AND APOJ BIOMARKER-BASED PREVENTION AND TREATMENT OF DEMENTIA

(76) Inventor: Jay L. Lombard, New City, NY (US)

(21) Appl. No.: 13/177,032

(22) Filed: Jul. 6, 2011

Related U.S. Application Data
(60) Provisional application No. 61/361,782, filed on Jul. 6, 2010.
(60) Provisional application No. 61/483,230, filed on May 6, 2011.

(51) Int. Cl. A61K 49/06; G01N 33/50; A61P 25/28; A61K 38/17

(52) U.S. Cl. 424/9.2; 514/17.8; 436/94

(57) ABSTRACT

Methods, kits, screens, assays, treatments and treatment regimes for treating a patient at risk for or suffering from dementia, and particularly Alzheimer's dementia which are based upon identification of enhanced risk by genotyping APOE and APOJ. In particular, described herein are methods for the prophylactic treatment of dementia based upon results of said genotyping. The systems and methods herein described may be used both to detect and to target the primary genetically mediated pathways associated with amyloid burden. For example, a system for deciding treatment based on previously identified genetic polymorphisms (e.g., APOE4, polymorphism in APOJ) that affect amyloid clearance is described herein; such patients may respond to treatments that modulate glial based GLT-1 (e.g., Tianeptine) and/or enhance HSP expression/activity (e.g., GGA).

### CROSS REFERENCE TO RELATED APPLICATIONS

This patent application claims priority to the following U.S. Provisional patent applications: Application No. 61/361,782, titled “TREATMENT OR PREVENTION OF DEMENTIA SYNDROMES BASED UPON MODULATION OF CLUSTERIN VIA HEAT SHOCK PROTEIN BASED THERAPEUTICS,” filed Jul. 6, 2010; and Application No. 61/483,230, titled “APOE4 AND APOJ BIOMARKER-BASED TREATMENT OF DEMENTIA,” filed May 6, 2011.

### INCORPORATION BY REFERENCE

All publications, patents, and patent applications mentioned in this specification are herein incorporated by reference in their entirety to the same extent as if each individual publication, patent or patent application was specifically and individually indicated to be incorporated by reference.

### FIELD OF THE INVENTION

Described herein are methods, kits, screens, assays, treatments and treatment regimes for treating a patient at risk for or suffering from dementia, and particularly Alzheimer's dementia. These methods, kits, screens, assays, treatments and treatment regimes are based a previously undisclosed theory describing risk for dementia in terms of an imbalance in amyloid accumulation and clearance which is described in detail herein. In practice, these methods, kits, screens, assays, treatments and treatment regimes may include the identification of an enhanced risk of developing or exacerbating dementia in an individual by genotyping markers which affect amyloid accumulation and/or clearance (e.g., APOE and APOJ). For example, described herein are methods for the prophylactic treatment of dementia based upon results of said genotyping. In part, it is herein proposed that pathogenic mechanisms mediated by polymorphisms in such genes may lead to protein accumulation. Accumulation and aggregation of disease-causing proteins is a hallmark of several neurodegenerative disorders, and the systems and methods herein described may be used both to detect and to target the primary genetically mediated pathways associated with amyloid burden.

Although many of the genes and proteins described herein are well known, the model for understanding accumulation and clearance of these proteins proposed herein provides new and unexpected methods and systems for detecting, preventing and treating dementia. For example, described herein are systems for deciding treatment based on previously identified genetic polymorphisms that affect amyloid clearance. In one variation, the model described herein predicts that patients having an APOE4 polymorphism, and/or polymorphisms APOJ (clusterin) may have impaired ability to clear amyloid due to defects in astrocytic removal of amyloid; on this basis we herein propose that even pre-symptomatic dementia may be treated with agents that enhance clearance of amyloid, such as heat shock protein (HSP) inducers like acyclic polyisoprenoid geranylgeranylacetone (GGA), and/or agents that modulate glutamate transport (GLT-1 activity) much as the GLT-1 modifying agent Tianeptine. Thus, the mechanisms described herein from the basis of a previously undisclosed method, system, and therapy to treat Alzheimer's dementia, particularly individuals having the genetic subtypes of APOE4 and or APOJ polymorphisms.

### BACKGROUND OF THE INVENTION

Alzheimer's disease is a neurodegenerative disease of the central nervous system associated with progressive memory loss resulting in dementia. An estimated 4.5 million Americans have Alzheimer's disease (“AD”). By 2050, the estimated range of AD prevalence will be 11.3 million to 16 million. Currently, the societal cost of AD to the U.S. is $100 billion per year, including $61 billion born by U.S. businesses. Neither Medicare nor most private health insurance covers the long-term care most patients need.

Alzheimer's disease is difficult to diagnose because appropriate diagnostic to shave not yet been identified. The ability to predict that an asymptomatic subject is at risk fix developing the disease is even more difficult. A method that would provide a better means by which Alzheimer's disease could be diagnosed, or the risk of developing the disease could be assessed, would be beneficial, as a therapeutic intervention could potentially be applied at an earlier stage of the disease process. This is especially important in relation to a neurodegenerative disease such as Alzheimer's; as it appears the underlying genetic and biochemical abnormalities are operative many years prior to the onset of identifiable symptoms.

Based on current population projections, it has been estimated that by 2050 the number of individuals over 65 will increase to 1.1 billion worldwide and as a consequence, the number of cases of dementia to 37 million. Faced with such an enormous public health and socio-economic burden the importance of therapeutic intervention aimed at either finding a cure or preventing disease progression cannot be overstated.

It is difficult to diagnose neurodegenerative disorders, such as dementia or any other progressive disorder, particularly in early stages of the disease, and virtually impossible to diagnose in pre diagnostic stages. The result of having better diagnostic or risk assessment tools would be the more timely administration of appropriate therapies, as well as more target-specific interventions. Also as discussed herein, if the genotype of a diseased patient is known, optimal therapies may be determined and administered to the patient, resulting in a faster recovery or even prevention from the disease. Thus, the application of gene targeted therapy has clinical relevance for both the prevention and treatment of disorders such as Alzheimer's.

Furthermore, the implementation of a presymptomatic AD treatment trial/surrogate marker development paradigm, as which will be subsequently disclosed, would make it possible to evaluate investigational presymptomatic treatments trials sooner than otherwise possible (i.e., using safety and tolerability data from fewer patients), since, it would provide the data needed to show the extent to which an established treatment's effects on different biomarkers predicts a clinical benefit.

To help bridge the gap between disease models and large and costly clinical trials with high failure rates, biomarkers for the intended biochemical drug effect are of considerable value.

Currently, the primary method of diagnosing AD in living patients involves taking detailed patient histories, administering memory and psychological tests, and ruling out other explanations for memory loss and cognitive defects, including temporary (e.g., depression or vitamin B₁₂ deficiency) or permanent (e.g., stroke) conditions. These clinical diagnostic methods, however, are not foolproof.

AD cannot be diagnosed with complete accuracy until after death, when autopsy reveals the disease's characteristic amyloid plaques and neurofibrillary tangles in a patient's brain. In addition, clinical diagnostic procedures are only helpful after patients have begun displaying significant, abnormal memory loss or personality changes. By then, a patient has likely had AD for years.

Amyloid deposition may be present for many years before the onset of clinical symptoms. As has been seen in the treatment of heart disease, even modest preventative treatments can have hugely significant clinical outcomes and drastically reduce disease prevalence. Thus, there are compelling reasons to evaluate promising treatments presymptomatically. Better diagnostics or risk assessment tools would allow more timely administration of appropriate therapies, as well as more target specific interventions.

Given the magnitude of the public health problem posed by AD, considerable research efforts have been undertaken to elucidate the etiology of AD as well as to identify biomarkers secreted proteins or metabolites) that can be used to diagnose and/or predict whether a person is likely to develop AD. Because the CNS is relatively isolated from the other organs and systems of the body, most research (in regards to both disease etiology and biomarkers) has focused on events, gene expression, biomarkers, etc. within the central nervous system when studying Alzheimer's dementia. With regards to biomarkers, the proteins amyloid beta and tau are probably the best characterized. Research has shown that cerebrospinal fluid (“CSF”) samples from AD patients contain higher than normal amounts of tau, which is released as neurons degenerate, and lower than normal amounts of beta amyloid, presumably because it is trapped in the brain in the form of amyloid plaques. Because these biomarkers are released into CSF, a lumbar puncture (or “spinal tap”) is required to obtain a sample for testing.

A number of U.S. patents have been issued relating to methods for diagnosing AD, including U.S. Pat. Nos. 4,728,605, 5,874,312, 6,027,896, 6,114,133, 6,130,048, 6,210,895, 6,358,681, 6,451,547, 6,461,831, 6,465,195, 6,475,161, and 6,495,335. Additionally, a number of reports in the scientific literature relate to certain biochemical markers and their correlation/association with AD, including Fahnestock et al., 2002, J. Neural. Transm. Suppl. 2002(62):241-52; Masliah et al., 1195, Neurobiol, Aging 16(4):549-56; Power et al., 2001, Dement. Geriatr. Cogn. Disord.

Genome-wide association studies (GWAS) have gained considerable momentum over the last couple of years for the identification of novel complex disease genes. In the field of Alzheimer's disease (AD), there are currently eight published and two provisionally reported GWAS, highlighting over two dozen novel potential susceptibility loci beyond the well-established APOE association. However, what is needed is a framework for understanding how to interpret these results, and in particular, how to apply an interpretation into a rational treatment regime. Described herein are methods and systems to address this need, including methods for classifying or stratifying a subject in a subgroup of a clinical trial or a therapy for the treatment of memory loss, memory preservation, MCI or Alzheimer's disease. As described in greater detail below, the methods described herein may include determining the genotype of said subject, particularly at the nucleotides encoding amino acids of the APOE gene and APOJ gene (which may include the promoter region, introns, exons, expressed forms, mRNA, etc.).

The subjects may be stratified into a subgroup for said clinical trial or therapy based upon their genotype in order to reduce likely amyloid burden and toxicity. For example, we propose evaluating promising Aβ-modifying treatments using biomarkers related to abnormal protein aggregation in Alzheimer's disease.

To date, genetic studies have identified three genes associated with dementia, including apolipoprotein E (“APOE”), which is the strongest and most common genetic risk factor for AD, but does not necessarily cause it. The Apolipoprotein E (APOE) locus (including SNPs such as rs2075650) is strongly associated with risk of developing dementia. In Alzheimer's disease, A_{beta} accumulates in brain due to altered CNS clearance of this protein.

Although 40-65% of AD patients have at least one copy of the APOE4 allele, APOE4 is not a determinant of the disease; at least a third of patients with AD are APOE4 negative and some APOE4 homozygotes never develop the disease. Yet those with two APOE4 alleles have up to 20 times the risk of developing AD. The ε4 allele of the apolipoprotein E (APOE) gene remains the most widely replicated genetic risk factor for late-onset AD, with ε4 carriers having a greater risk (3-15-fold), as well as an earlier age of disease onset.

Of the three common human apolipoprotein (APO) E isoforms (APOE2, APOE3, and APOE4), APOE4 is the major genetic risk factor for Alzheimer's disease (AD). All other candidate genes identified in numerous genetic screens of AD populations fall far short of APOE4 for statistical impact on AD pathogenesis. In a typical control population, approximately 20% of the individuals carry at least one APOE4 allele. That percentage would rise to 65% in non-related patients with sporadic AD and to 80% in those with familial AD. This impact is more sobering given the emerging evidence that current therapies, including those targeting Aβ, are relatively ineffective in APOE4 carriers and that there has been a general lack of activity in developing APOE4-targeted therapies. Indeed, APOE4 is a viable drug target for treating AD.

In brain, APOE is mainly synthesized and secreted by astrocytes and microglia both of which are found to surround amyloid plaques. Astrocytes promote Aβ clearance via an APOE-dependent mechanism.

Both in vitro and in vivo studies demonstrate that APOE4 reduces astrocyte Aβ clearance. The ability of macrophages to degrade Aβ is APOE isoform-dependent (E2>E3>E4), the APOE isoform-dependent macrophage-mediated Aβ degradation in part, mediated by secretion of matrix metalloproteinase-9 (MMP-9), indicating that the brain requires immune mediated pathways to effectively remove amyloid. Hippocampal Aβ immunoreactivity is reduced by >80-90% alter incubation for 96 h with APOE2 or e3 polymorphisms in mouse macrophages and only by ˜60% when incubated with APOE4-d macrophages. Astrocytes degrade beta-amyloid (A_{beta}) via MMP-9. APOE4 significantly dampens A_{beta}-induced MMP-9 levels, and reduction of astrocytic MMP-9 by APOE4 may affect A_{beta} clearance and promote A_{beta} deposition in AD.

Thus, it is herein proposed, in part, that in APOE4 individuals, lower expression of MMP-9 may result in reduced A_{beta} clearance.

Macrophage-mediated Aβ degradation through MMP-9 expression in brain may constitute a peripheral clearance mechanism and delineates a previously unknown role for APOE in modulating Aβ-degrading proteases that may help explain the role of APOE as a genetic risk factor for AD. APOE isoform-dependent difference in the ability of macrophages to efficiently degrade Aβ. APOE4 significantly dampens A_{beta}-induced MMP-9 levels, and reduction of astrocytic MMP-9 by APOE4 may affect A_{beta} clearance and promote A_{beta} deposition in AD.

The pathological accumulation of amyloid in APOE4 genotypes results in excess glutamate and neurodegeneration secondary to impaired glial GLT-1 activity. Aβ(1-40) induces a marked decrease in glutamatergic transporters (GLAST and GLT-1) expression. In transgenic animal models of Alzheimer's disease, levels of the astrocyte-specific glutamate transporter, GLT1, are lower than in WT mice.

AβPP23 mice, plaque formation and gliosis in cortex and hippocampus is accompanied by decreased GLT-1 expression supporting the hypothesis that alterations in GLT-1 may be involved in AD pathogenesis.

Glial glutamate transporter (GLT-1) variants are significantly down-regulated in murine models of neurodegenerative diseases and GLT-1 expression is absent in the inclusions observed in protein aggregation. In mice expressing human APOE isoforms, a significant loss of GLT-1 is demonstrated.

in contrast, cellular up regulation of HSP70 expression provides cytoprotection against Aβ. HSP70 activity in relation to inhibition of Aβ oligomerization and stimulation of Aβ phagocytosis, suggesting that stimulation of the expression of HSP70 could prove effective in the treatment of AD. Transgenic mice expressing HSP70 display lower levels of Aβ, Aβ plaque deposition, confirming the potential therapeutic benefit of HSP70 for the prevention or treatment of AD.

Apolipoprotein J (clusterin) is a ubiquitous multifunctional glycoprotein capable of interacting with a broad spectrum of molecules, including amyloid, and functions as a heat shock protein involved amyloid degradation.

Previous biological studies support roles of APOJ in the clearance of amyloid (A_{beta}) peptide. Endocytic response associated with the accumulation of clusterin/APOJ protein suggests that clusterin/APOJ has a role in the clearance of amyloid-beta peptides by binding soluble beta-amyloid. Extracellular APOJ facilitates the conversion of diffuse A_{beta} deposits into amyloid and enhances tau phosphorylation neurites surrounding these plaques. SNPs at the CULT (also known as APOJ) gene (e.g., rs11136000) have been associated with dementia, and it is herein proposed that this increased risk is due to defective heat shock protein mediated clearance of amyloid.

APOJ is present in amyloid plaques and may represent a defense response against local damage to neurons via binding to hydrophobic regions of partially unfolded, stressed proteins, therefore avoiding aggregation in a chaperone-like manner.

Clusterin/APOJ may therefore be understood as a secreted chaperone and the endocytic response associated with the accumulation of clusterin/APOJ protein suggests that clusterin/APOJ has a role in the clearance of amyloid-beta peptides. The relationship of genetic polymorphisms of clusterin and risk of dementia strongly suggests that this may represent a critical pathway of the disease and efforts to modify the activity of clusterin as a novel therapeutic target.

Research into possible mechanisms leading to the accumulation of modified Tau protein and the possibility of removing Tau protein from the system have revealed that clusterin can interact with Tau and mediate its degradation. Hsp70/Hsc70, a member of the chaperone protein family, interacts with Tau protein and mediates proper folding of Tau to promote its degradation.

In Alzheimer's disease, a hyperphosphorylated form of the protein tau (p-tau) forms intracellular inclusions known as neurofibrillary tangles. Deposits of p-tau have also been found in the brains of patients with Alzheimer's and in CSF. Ubiquitinated tau is one component in neurofibrillary tangles (NFTs), which are a major histopathological feature of Alzheimer's disease. The accumulation of tau and amyloid beta proteins is the major molecular pathology of Alzheimer's disease. The mechanisms leading to the accumulation of these proteins are related to genetic polymorphisms in heat shock protein function.

Expediting the removal of these p-tau species may be a relevant therapeutic strategy and represents another previously undisclosed application for genetic or biomarker testing and subsequent administration of heat shock protein inducers in the treatment of dementia.

Molecular chaperones and heat shock proteins (HSP) have emerged as critical regulators of proteins associated with neurodegenerative disease pathologies. Amyloid precursor protein (APP), members of the gamma-secretase complex (presenilin 1 [PS1] collectively), and the microtubule-associated protein tau (MAPT) are all in contact with chaperones, and the function of heat shock proteins is to facilitate the removal of these abnormal proteins.

The molecular chaperone, heat shock protein 70 (Hsp70), acts at multiple steps in a protein's life cycle, including during the processes of folding, trafficking, remodeling and degradation. Heat-shock proteins (HSPs) are stress-induced chaperones that facilitate the refolding and, thus, the degradation of abnormal proteins.

Endocytic response associated with the accumulation of clusterin/APOJ protein suggests that clusterin/APOJ has a role in the clearance of amyloid-beta peptides.

Clusterin has chaperone activity in vitro. Clusterin inhibits stress-induced precipitation of a very broad range of structurally divergent protein substrates, binds irreversibly via an ATP-independent mechanism to stressed proteins to form solubilized high molecular weight complexes, and stabilizes stressed proteins in a state competent for refolding by heat shock protein 70 (HSP70). Furthermore, clusterin inhibits stress-induced precipitation of proteins. The demonstration that clusterin can stabilize stressed proteins in a refolding-competent state suggests that, during stresses, the action of clusterin may inhibit rapid and irreversible protein precipitation and produce a reservoir of inactive but stabilized molecules from which other refolding chaperones can subsequently salvage functional proteins. Hsp-70, a chaperone protein, has been shown to bind both these proteins and regulate their degradation.

Genetic studies have strongly linked Hsp70 and its co-chaperones to neurodegeneration, yet the potential of this chaperone as a therapeutic target remains largely underexplored, and represents a potential and therapeutic target for dementia. Further, therapies which target clusterin in efforts to improve its function as a treatment for dementia has been previously undisclosed. In addition, methods of modifying the expression of APOJ and/or APOE in genetically vulnerable individuals based upon up-regulation of heat shock pro ins has not been previously disclosed as a treatment to prevent or treat dementia. Modification of the expression of the APOJ and APOE gene, by pharmaceutically up-regulating chaperone proteins which can reduce amyloid and tau protein aggregation, represents a novel way of treating people at risk of dementia who exhibit polymorphisms in the APOJ/clusterin gene and or APOE4 polymorphisms.

The accumulation of amyloid beta proteins as a result of genetically mediated clusterin down regulation may represent a major molecular pathology of Alzheimer's disease. The mechanisms leading to the accumulation of these proteins are not completely clear but are likely related to impairments of normal heat shock protein chaperone activity.

As described herein, modification of the expression of the APOJ gene, by pharmaceutically up-regulating chaperone proteins, may reduce amyloid and tau protein aggregation, and represents a novel way of treating people at risk of dementia who exhibit polymorphisms in the APOJ/clusterin gene. Compounds or compositions that can induce or increase expression of a heat shock protein are therefore included in this invention, particularly those linked to up regulation of heat shock proteins, and HSP 70 in particular.

Thus, we herein hypothesize that patient's having the APOE4 and/or clusterin polymorphisms may have a problem in excessive deposition and/or clearance of amyloid A_{beta}) and tau proteins. The preferred treatment in these patients includes treatment with up-regulators of HSPs (e.g., GGA) and/or Oat activity (e.g., Tianeptine). In these patients anti-inflammatory treatments, which may potentially inhibit requisite immune mediated amyloid clearance, may be contraindicated. This hypothesis is supported by observations that the Apolipoprotein APOE4 allele (APOE4) patients have lower C-reactive protein (CRP) than those without APOE4.

CRP is significantly lower among those with APOE4 than in those without. Among those with APOE4, CRP was associated with lower rates of dementia. Lower CRP in those with APOE4 may reflect immune effects of the APOE4 genotype. Higher CRP in those with APOE4 may be a marker of better immune function, leading to tower rate of dementia and AD. Studies which have examined the use of anti-inflammatory agents to treat or protect against AD, have had mixed results. For example, despite early experimental studies suggesting that nonsteroidal anti-inflammatory drugs (NSAIDs) may protect against Alzheimer disease (ND), clinical trials and other observational studies, including the Adult Changes in Thought (ACT) study, showed no protection or promotion of AD. Such results have lead many to conclude that anti-inflammatory drugs do not prevent dementia in AD patients. The framework proposed herein suggests that these contradictory conclusions may arise because these studies did not account for the different genetic based influence on the etiologies of dementia and AD in the patient populations; for example, patient's with APOE4 polymorphisms may be negatively affected by the use of NSAIDs or other anti inflammatory targeted treatments as these pathways may actually be required to clear amyloid through the heat shock protein pathway.

Thus, described herein are improved methods and systems for determining if a patient is at an increased risk of developing dementia (e.g., Alzheimer's dementia), and meth ds of determining the most appropriate treatment of dementia, based on the collective results of the patient's genetic profile.

Although a link between inflammation and Alzheimer's has long been proposed, to date the analysis has failed to understand the complex and multidimensional rote of both pro- and anti-inflammatory agents in Alzheimer's dementia. Genes implicated in handling neural inflammation, and particularly those linked to Alzheimer's, may be used to help determine effective prophylactic treatment. In this regard, it is currently disclosed that individuals at risk of dementia who express APOE4 and/or APOJ have reduced immune capacity to up-regulate heat shock proteins and/or respond to reduced GLT-1 activity. These pathogenic mechanisms result in defective astrocytic clearance of amyloid and the pathological accumulation of protein aggregates.

Thus, we herein describe therapies and treatments (including therapeutic compounds and compositions) that my be specifically used to prophylactically treat a patient for Alzheimer's dementia. The methods and systems described herein may include proposing or guiding patient treatment based upon a patient's genetic risk factors, and that these factors indicate reduced astrocytic removal of amyloid and other abnormal protein aggregates. The use of these genetic abnormalities as a determinant of specific treatments has not previously been described, and can guide treatment (and especially prophylactic treatment) by enhancing amyloid removal by up-regulating HSPs (e.g., HSP-70) and/or preventing excess amyloid accumulation via administration of a glt-1 modifier, such as Tianeptine. In particular, the methods and systems described herein may determine an optimal treatment using agents such as GGA and Tianeptine.

### SUMMARY OF THE INVENTION

The present invention relates to methods, kits, screens, assays, treatments and treatment regimes for treating a patient at risk for or suffering from dementia (e.g., dementia associated with Alzheimer's disease). In particular, described herein are methods and systems for prophylactically treating people at risk for dementia. In general these methods (and systems, screens, kits, treatments, treating regimes and/or assays for performing them) may include: (1) determining a patient's genotype related to genes related to susceptibility for dementia, and particularly Alzheimer's; and (2) characterizing the patient as having enhanced risk of amyloid production and/or impaired amyloid clearance (e.g., positive for APOE4 and/or clusterin), and therefore, the patient may be determined to preferably respond to agents which induce heat shock proteins and/or agents which modulate GLT-1 activity/expression by astrocytes).

Although many different genes have been linked to an increase in genetic susceptibility for dementia (and particularly Alzheimer's dementia), APOE and to a lesser extent, APOJ, are of particular interest.

APOE subtypes may have different vulnerability to dementia and, as hypothesized herein, may therefore have differential responses to drugs.

We herein hypothesize that APOE4 individuals, who are generally considered to be at the highest risk of developing dementia, are actually those for which using anti-inflammatory agents may be counterproductive. Thus, these patients may be regarded as those for whom prophylactic treatment with agents which modulate astrocyte function, and in particular astrocyte GLT-1 activity, may prevent or inhibit the development of Alzheimer's dementia. These astrocyte modulating agents may include heat-shock protein inducing agents, such as Geranylgeranylacetone (GGA) and agents which modulate GLT-1 level or activity, such as Tianeptine.

In patient's having the APOE4 polymorphism and possibly a clusterin polymorphism (e.g., the rs11136000 polymorphism in clusterin gene), the pathogenesis of amyloid burden may be due to failure of normal immunogenic mechanisms necessary to degrade and clear amyloid. Thus, it is advisable to enhance, rather than inhibit, pro-inflammatory pathways associated with glial cell function. As mentioned, this may include increasing the expression of heat shock proteins using agents such as GGA, or either synergistically, to enhance astrocyte GLT-1 function with agents such as Tianeptine.

### BRIEF DESCRIPTION OF THE DRAWINGS

FIG. 1A illustrates one theoretical model for dementia (“the double-hit” model or hypothesis) based on the regulation of amyloid protein as described herein. FIG. 1B illustrates the theoretical model of FIG. 1A indicating theoretical points for therapeutic intervention by an up-regulator or HSP activity or expression (e.g., GGA), and by modulation of the GLT-1 glutamate transporter (Tianeptine).

### DETAILED DESCRIPTION OF THE INVENTION

In some variations, the methods and systems described herein may be used to determine prophylactic Alzheimer's dementia treatment categories based upon results of genetic testing which indicates heightened dementia risk secondary to excess amyloid accumulation and/or attenuated glial based removal of protein aggregates.

In particular, described herein are methods for prophylactically treating a patient for Alzheimer's with agents that either up-regulate HSPs/chaperons (e.g., HSP-70 up-regulators/enhancers such as GGA) and/or agents that modulate GLT-1, such as Tianeptine), particularly if the patient has an APOE4 and/or a Clustrin polymorphism. Such patient's may be determined to be dependent on glial based pathways to inhibit or remove pathological accumulation of protein aggregates such as amyloid, and are predicted to respond preferentially to these glial based therapeutic treatments which will help with reducing amyloid deposition and/or the clearance of amyloid. For example, these patients may be treated with Geranylgeranylacetone (GGA) and/or Tianeptine.

Patients that have either (or both) the APOE4 or a mutation in clusterin (e.g., the rs11136000 polymorphism in clusterin gene) may be referred to as possessing excess amyloid accumulation and/or reduced astrocytic mediated amyloid removal, and that the therapy may be directed to inducing up-regulation of HSP expression. For example, these patients may be treated with Geranylgeranylacetone (GGA) and/or Tianeptine. Thus, patients having the APOE4 and/or clusterin polymorphism(s) may be treated with HSP inducers such as GGA as a preferred neuroprotective agent or a GLT-1 modulator, such as Tianeptine

For example, the first group or classification of at-risk patients may include those with APOE4 homozygotes, and/or those having a clusterin polymorphisms (e.g., rs 11136000).

The methods and systems described herein may be directed towards determining the appropriateness and/or effectiveness of a specific, disease modifying therapy in a patient or population of patients, e.g., by determining risk of developing or further developing a neurodegenerative disorder linked to the specific genetic expression of one or some combination of APOE and Clusterin. Polymorphisms in one or more of these genes may alter protein expression in the brain. Further, as will be demonstrated, these genes represent unique primary pathological changes which therefore require specific and unique selective pharmacological interventions. In the patients expressing the APOE4 and/or Clusterin (APOJ) polymorphism, reduced amyloid degradation may occur secondary to impaired immune based astrocytic proteolytic processes.

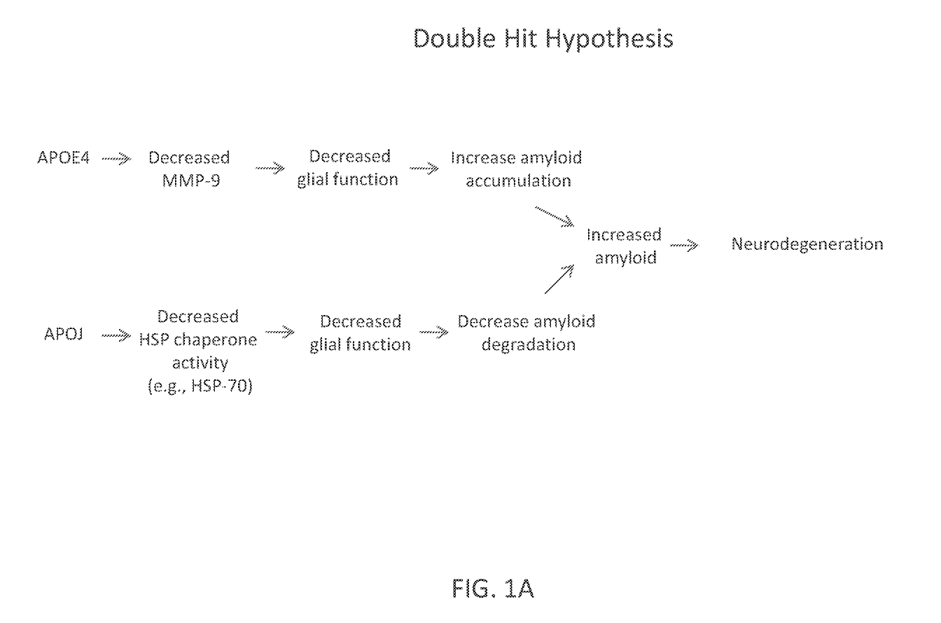

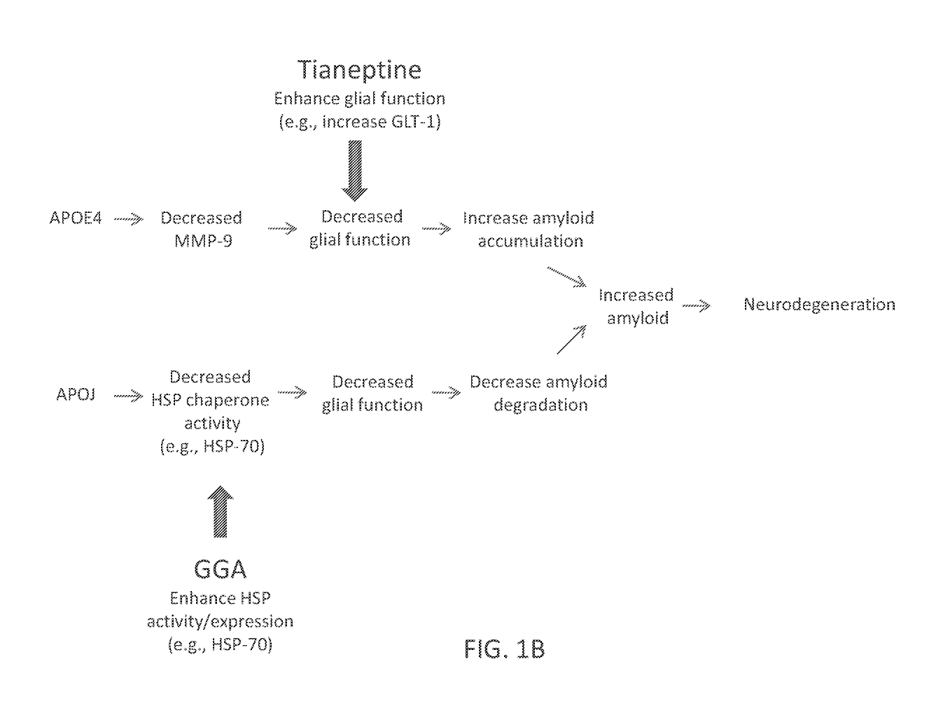

Polymorphisms in the APOE gene are widely regarded as the most important genetically based risk factor for late onset dementia. Individuals heterozygous for the APOE4 gene are at fourfold higher risk and homozygotes, 10× risk. However, the relationship of APOE4 polymorphisms to excess amyloid deposition remains unclear. Apolipoprotein E4 (APOE4), the most prevalent genetic risk factor for Alzheimer's disease, is histopathologically associated with increased deposition of amyloid-beta. It is currently proposed that the APOE4 gene polymorphism results in reduced endogenous immune passed pathways, such as MMP-9, to degrade amyloid. For example, APOE4 and APOJ biomarkers may lead to enhanced amyloid deposition due to reduced glial-based proteolytic clearance, as illustrated in FIG. 1A.

Amyloid beta-peptide (A_{beta}) clearance from the central nervous system (CM) maintains its low levels in brain. In Alzheimer's disease, A_{beta} accumulates in brain possibly because of altered CNS clearance of this protein. It now appears that altered clearance of amyloid and other CNS specific proteins is related to genetic abnormalities in heat shock proteins which function as facilitators of protein aggregation removal in the brain. Abnormal protein accumulation resulting from genetic impairments in clusterin is a hallmark of dementia and efforts to enhance degradation of these proteins is a high value target and a previously undisclosed aspect of this invention.

The pathophysiology of reduced protein degradation in dementia may be related to genetic polymorphisms in the ubiquitin degradation system. This system may depend upon heat shock proteins which transport abnormal protein complexes to the endoplasmic reticulum, where they are processed by proteasomes, degraded and cleared.

GLT-1 is a transporter protein preferentially expressed in astrocytes. Evidence that GLT-1 chaperone activity is also linked to Alzheimer's includes the relative absence of this protein in pathological inclusions. Further, reduced GLT-1 has been associated with the APOE4 genotype.

We herein propose that in individuals who display polymorphisms in these chaperone proteins, i.e. heat shock protein pathways, including and specifically related to the clusterin gene, and GLT-1 function related to APOE4, there is consequential excessive protein aggregation and higher risk of developing dementia or other neurodegenerative conditions. These individuals, when appropriately identified by gene testing, are most suitable to receive tong term treatment with a HSP inducer and/or GLT-1 inducer, as described in the paragraphs below.

The accumulation of amyloid beta proteins as a result of genetically mediated clusterin down regulation may represent a major molecular pathology of Alzheimer's disease (AD). The mechanisms leading to the accumulation of these proteins are not completely clear but are likely related to impairments of normal heat shock protein chaperone activity.

Apolipoprotein (clusterin) is a ubiquitous multifunctional glycoprotein capable of interacting with a broad spectrum of molecules, including amyloid, and functions as a heat shock protein involved amyloid degradation. Previous biological studies support roles of APOJ the clearance of amyloid (A_{beta}) peptide. Endocytic response associated with the accumulation of clusterin/APOJ protein suggests that clusterin/APOJ has a role in the clearance of amyloid-beta peptides by binding soluble beta-amyloid. Extracellular APOJ facilitates the conversion of diffuse A_{beta} deposits into amyloid and enhances tau phosphorylation in neurites surrounding these of plaques. SNPs at the CLU (also known as APOJ) gene (e.g., rs11136000) have been associated with dementia, and this increased risk is due to defective heat shock protein mediated clearance of amyloid.

APOJ is present in amyloid plaques and may represent a defense response against local damage to neurons via binding to hydrophobic regions of partially unfolded, stressed proteins, therefore avoiding aggregation in a chaperone-like manner. Clusterin/APOJ, a secreted chaperone and the endocytic response associated with the accumulation clusterin/APOJ protein suggests that clusterin/APOJ has a role in the clearance of amyloid-beta peptides. The relationship of genetic polymorphisms of clusterin and risk of dementia strongly suggests that this may represent a critical pathway of the disease and efforts to modify the activity of clusterin may provide a novel therapeutic target.

Hsp-70, a chaperone protein, has been shown to bind defective proteins such as beta amyloid and tau and regulate their degradation. Molecular chaperones and heat shock proteins (HSPs) have emerged as critical regulators of proteins associated with neurodegenerative disease pathologies. Amyloid precursor protein (APP), members of the gamma-secretase complex (presenilin 1, or PS1, collectively), the microtubule-associated protein tau (MAPT) are all in contact with chaperones, and the function of chaperones/heat shock proteins is to facilitate the removal of these abnormal proteins.

The molecular chaperone, heat shock protein 70 (Hsp70), acts at multiple steps in a protein's life cycle, including during the processes of folding, trafficking, remodeling and degradation. Heat-shock proteins (HSPs) are stress-induced chaperones that facilitate the refolding and therefore the degradation of abnormal proteins. Thus, novel methods to stimulate HSP may represent a method to enhance clearance of protein aggregation characteristic of neurodegenerative diseases such as Alzheimer's dementia.

Clusterin has chaperone activity in vitro. Clusterin inhibits stress-induced precipitation of a very broad range of structurally divergent protein substrates, binds irreversibly via an ATP-independent mechanism to stressed proteins to form solubilized high molecular weight complexes, and stabilizes stressed proteins in a state competent for refolding by heat shock protein 70 (HSP70). Furthermore, clusterin inhibits stress-induced precipitation of proteins and can stabilize stressed proteins in a refolding-competent. Hsp-70, a chaperone protein, has been shown to bind both these proteins and regulate their degradation. Genetic studies have strongly linked Hsp70 and its co-chaperones to neurodegeneration, yet the potential of this chaperone as a therapeutic target remains largely underexplored, and represents a potential and therapeutic target for dementia.

Methods of modifying the expression of APOJ in genetically vulnerable individuals based upon up-regulation of heat shock proteins has not yet been described as a treatment to prevent or treat dementia characterized by protein miss-folding and protein aggregation.

Modification of the expression of the APOJ gene, by pharmaceutically up-regulating chaperone proteins, can reduce amyloid and tau protein aggregation, and represents a novel way of treating people at risk of dementia who exhibit polymorphisms in the APOJ/clusterin gene polymorphism as described. Compounds or compositions that can induce or increase expression of a heat shock protein relevant to this invention have also been linked to up regulation of heat shock proteins, and HSP 70 in particular.

Hsp70 is released from astrocytes and may activate matrix metalloproteinase-9 (MMP-9) gene expression to enhance proteolytic degradation of amyloid. Further, immunodepletion of Hsp70 abolishes its effect on MMP-9 expression. Taken together, these results suggest that extracellular Hsp70 induces the expression of MMP-9, which may mediate proteolytic degradation of miss-folded proteins.

The pathophysiology of protein aggregation in Alzheimer's may be due to genetic polymorphisms in the ubiquitin degradation system. This system depends upon heat shock proteins which transport abnormal protein complexes to the endoplasmic reticulum, where they are processed by proteasomes, degraded and cleared. In individuals who display polymorphisms in heat shock protein pathways, including and specifically related to the clusterin gene, there is consequential excessive protein aggregation and higher risk of developing dementia or other neurodegenerative conditions. Altered clearance of amyloid and other CNS specific proteins may be related to genetic abnormalities in heat shock proteins which function as facilitators of protein aggregates in the brain. Heat shock proteins may also function as chaperones to remove abnormal protein aggregates in the brain. Abnormal protein accumulation is a hallmark of dementia and efforts to enhance degradation of these proteins is a high value target and a (previously undisclosed aspect of this invention.

In subjects having a polymorphism in the CLU gene (clusterin, apolipoprotein J), the accumulation of tau and amyloid beta proteins may be the result of genetically mediated clusterin down-regulation may represent a major molecular pathology of Alzheimer's. The mechanisms leading to the accumulation of these proteins are not completely clear but are likely related to clusterin mediated impairments of normal heat shock protein chaperone activity.

In Alzheimer's disease (AD), a hyperphosphorylated form of the protein tau (p-tau) forms intracellular inclusions known as neurofibrillary tangles. Deposits of p-tau have also been found in the brains of patients with Alzheimer's and in CSF. Ubiquitinated tau is one component neurofibrillary tangles (NFTs), which are a major histopathological feature of Alzheimers disease. The accumulation of tau and amyloid beta proteins is the major molecular pathology of Alzheimer's disease (AD). The mechanisms leading to the accumulation of these proteins are related to genetic polymorphisms in heat shock protein function. Hsp-70, a chaperone protein, has been shown to bind both these proteins and regulate their degradation. Research into possible mechanisms leading to the accumulation of modified Tau protein and the possibility of removing Tau protein from the system have revealed that heat shock proteins can interact with Tau and mediate its degradation under normal circumstances. Hsp70/Hsc70, a member of the chaperone protein family, interacts with Tau protein and mediates proper folding of Tau and which can promote degradation of Tau protein. Expediting the removal of these p-tau species may be a relevant therapeutic strategy and represents another previously undisclosed application for genetic or biomarker testing and subsequent administration of heat shock protein inducers in the treatment of dementia.

Molecular chaperones and heat shock proteins (HSP) have emerged as critical regulators of proteins associated with neurodegenerative disease pathologies amyloid precursor protein (APP), members of the gamma-secretase complex (presenilin 1 [PS1] collectively), the microtubule-associated protein tau (MAPT) are all in contact with chaperones and the function of heat shock proteins is to facilitate the removal of these abnormal proteins.

The molecular chaperone, heat shock protein 70 (Hsp70), acts at multiple steps in a protein's life cycle, including during the processes of folding, trafficking, remodeling and degradation.

Endocytic response associated with the accumulation of clusterin/APOJ protein suggests that clusterin/APOJ has a rote in the clearance of amyloid-beta peptides. Clusterin has chaperone activity in vitro. As mentioned, clusterin inhibits stress-induced precipitation of a very broad range of structurally divergent protein substrates, binds irreversibly via an ATP-independent mechanism to stressed proteins to form solubilized high molecular weight complexes, and stabilizes stressed proteins in a state competent for refolding by heat shock protein 70 (HSP70). Furthermore, the demonstration that clusterin can stabilize stressed proteins in a refolding-competent state suggests that, during stresses, the action of clusterin may inhibit rapid and irreversible protein precipitation and produce a reservoir of inactive but stabilized molecules from which other refolding chaperones can subsequently salvage functional proteins.

Genetic studies have strongly linked Hsp70 and its co-chaperones to neurodegeneration, yet the potential of this chaperone as a therapeutic target remains largely underexplored, and represents a potential and therapeutic target for dementia. Further, therapies which target clusterin in efforts to improve its function as a treatment for dementia have not been previously disclosed. Methods of modifying the expression of APOJ and/or APOE in genetically vulnerable individuals based upon up-regulation of heat shock proteins have not been previously disclosed as a treatment to prevent or treat dementia which is characterized by protein miss-folding and protein aggregation. Modification of the expression of the APOJ and APOE gene, by pharmaceutically up-regulating chaperone proteins which can reduce amyloid and tau protein aggregation, represents a novel way of treating people at risk of dementia who exhibit polymorphisms in the APOJ/clusterin gene polymorphisms.

Individuals identified by single nucleotide polymorphisms related to dementia risk, or identified by other state of the art means which demonstrate abnormal protein accumulation in the brain (such as tagged protein radiotracer studies) hereby disclosed as being candidates for the administration of an agent which restores impaired protein degradation. For example, compounds; e.g., GGA and Tianeptine, that up-regulate heat shock proteins or modulate GLT-1 expression, can potentially correct the risk of developing dementia and/or inhibiting its progression, by promoting the degradation of abnormal proteins associated with dementia.

### Model

FIG. 1A summarizes one variation of a novel model of the regulation of the amyloid clearance, summarizing some of the information provided above. In this model, which may be referred to as the “double hit” model or hypothesis, amyloid clearance is modulated by the level of MMP-9, which may be regionally expressed and regulated; MMP-9 may help degrade amyloid protein. Expression or activity of MMP-9 may be linked to APOE; those having one or more of the APEO4 variants of APOE may have a decreased level and/or activity of MMP-9, and therefore may have a decrease in astroycte clearance of amyloid.

Similarly, a decrease in HSP chaperone activity may also lead to a decrease in Astrocyte clearance of amyloid, as discussed above. Polymorphisms APOJ (e.g., rs11136000, rs17466684, rs2279590, rs1532278, rs1532277, Rs17466684, etc) may result in a decrease in HSP chaperone activity and therefore a decrease in clearance of amyloid. This decrease in clearance of amyloid therefore results in an increase in amyloid.

Based on this model, the effects of increased amyloid may be triggered and/or exacerbated by the reduction (even partial reduction) in either or both APOJ activity or expression and APOE4-MMP-9 activity or expression, which may result from polymorphic forms of these genes, their expression levels, and the proteins they encode. Thus it may be possible to determine if a patient is at risk based on the presence of one or more polymorphisms in the APOE (e.g., APOE4 form and/or APOJ. In some variations the level of elevation of the risk may be determined by the presence of either particular polymorphisms or the presence of multiple polymorphisms (or copies of a polymorphism), or both.

Once an elevated level of risk for dementia, based on a polymorphism in APOJ and/or the presence of APOE4 has been confirmed, an at-risk patient may be treated as illustrated in FIG. 1B, by the application of one or more agents that either enhances HSP activity and/or expression (e.g., GGA), and/or the application of one or more agents that modulate expression of GLT-1 and/or GLAST using Tianeptine.

In some variations, patients having the double hit of both APOE4 (e.g., decreased glial activity, and particularly decreased glial MMP-9 activity) and on APOJ polymorphism resulting in decreased HSP/chaperone activity (e.g., HSP-70 activity) may be particularly well suited to treatment with both an enhancer of HSP activity (e.g., GGA) and an enhancer of glial activity (e.g., Tianeptine). Such patient's may be categorized as high genetic risk for developing dementia. Patient's having a single hit (e.g., either a polymorphism in APOJ or a APOE polymorphism) may be categorized as intermediate (e.g., medium) risk, while patient's without any polymorphism APOE or APOJ may be regarded as having lower genetic risk. Gradation in risk may be assessed based on the number of alleles having a polymorphism, and/or the number of polymorphisms (e.g., multiple polymorphism in APOJ and/or APOE). Generally, the greater number of polymorphic alleles, the higher the risk. The assessed level of risk based on the genetic information may provide guidance on the therapy; for example, the use of a particular composition e.g., containing one or both of Tianeptine and/or GGA), the dosing regime, or the like.

### Enhancement of HSTs

Geranylgeranylacetone (GGA), is a heat shock protein inducing agent, which may have therapeutic application to individuals at risk of dementia who exhibit polymorphisms clusterin, a heat shock protein vital to amyloid and hyperphosphorylated tau degradation. GGA is one of a number of HSP enhancers. Teprenone (or geranylgeranylacetone) is a drug used for the treatment of gastric ulcers sold exclusively in Japan under the brand name SELBEX. Geranylgeranylacetone (GGA), a nontoxic antiulcer drug, has been shown to potently induce HSP expression in various tissues, including the central nervous system. In a cell model, GGA increased the levels of Hsp70, Hsp90, and Hsp105 and inhibited cell death. Oral administration of GGA also up-regulated the expression of HSPs in the central nervous system, promotes the expression of HSP70 in the brain, and therefore GGA may play an important role neuroprotection based upon its effects in individuals with abnormal clusterin function and reduced ability to mediate pro degradation. geranylgeranylacetone (GGA), a potent HSP inducer reduces amyloid oligomer levels and aggregates.

Small HSPs which are induced by GGA can directly interrupt amyloid oligomer formation, and the in vivo protective effects of the small HSPs induced by GGA administration on the development of abnormal protein aggregation associated with neurodegenerative diseases has been previously undisclosed as a therapeutic use of the compound for individuals displaying polymorphisms in the clusterin or APOE4 gene. Oral administration of GGA results in up-regulation of the expression level of HSP70. These effects GGA make it an ideally suited candidate to prevent or eat memory disorders associated with abnormal protein aggregation in individuals at risk of developing dementia, or who already have the disease, based upon the genetic polymorphism related to clusterin or APOE4 polymorphisms.

GGA, as a heat shock protein inducer, may correct impaired protein degradation associated with individuals at risk of developing or who already have symptoms of dementia. The administration of GGA will allow the up-regulation of degradation processes associated with the ubiquination-mediated degradation to facilitate amyloid degradation. In other words, one of the primary processes associated with Alzheimer's is the inability to degrade amyloid fibrils and tau, as a result of impaired protein ubiquination degradation processes. By identifying these individuals, ideally in (presymptomatic or in early stages of the disease, the administration of an agent like GGA will correct the degradation process by up-regulation of key heat shock proteins which are associated with the protein ubiquination process.

SELBEX (geranylgeranylacetone or GGA), has the formula:

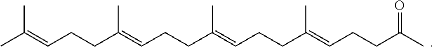

SELBEX was first marketed by Eisai Co., Ltd. in Japan in 1994 for the treatment of peptic ulcers. SELBEX, its synthesis, and its formulations are described in U.S. Pat. No. 4,169,157, which is incorporated herein by reference in its entirety.

SELBEX can be synthesized according to the method described in U.S. Pat. No. 4,169,157. Nonproprietary names of SELBEX are teprenone and geranylgeranylacetone, and its chemical name is 3:2 (5E:5Z) geometrical mixture of (9E_{;}13E)-6,10,14,18-tetramethyl-5,9,13,17-nonadecatetraen-2-one. The molecular formula of SELBEX is C₂₃H₃₈O, giving it a molecular weight of 330.55.

SELBEX can be administered according to the methods of the invention in any form suitable for oral administration, including capsule, powder, tablet, granule, pill, or liquid forms. In addition, SELBEX can be administered parenterally by injection or it can be administered as a suppository. Many suitable formulations are described in U.S. Pat. No. 4,169,157. A preferred formulation is a capsule that includes 50 mg SELBEX and, as inactive ingredients, tocopherol, sodium lauryl sulfate, and, if desired, pharmaceutically acceptable agents to provide color FD&C Blue No. 1 and FD&C Yellow No. 6). Another preferred formulation consists of fine granules, in which each gram of white to yellowish granules contains 100 mg of SELBEX. Extended release preparations are also claimed and are known methods to those skilled in the art.

Pharmaceutical compositions of the present invention can be administered by any means that achieve their intended purpose. For example, administration can be by parenteral, subcutaneous, intravenous, intramuscular, intraperitoneal, transdermal, buccal, or ocular routes. Alternatively, or concurrently, administration can be by the oral route. The dosage administered will be dependent upon the age, health, and weight of the recipient, kind of concurrent treatment, if any, frequency of treatment, and the nature of the effect desired.

SELBEX can be used according to the methods of the invention in a purely prophylactic mode, in which a person self-administers, for example, between about 20 mg to about 200 mg (e.g., 100 mg) of SELBEX orally.

Because GGA is practically insoluble in water, significant bioavailability can be problematic. Moreover, GGA may be taken three times a day with meals. Thus, there is a need for formulations which overcome these and other problems associated with the use of GGA. The present invention also contemplates controlled release compositions of GGA which eliminate the need for frequent administration.

For example, a GGA composition may comprise at least one surface stabilizer adsorbed on or associated with the surface of the GGA particle which can overcome the poor bioavailability of conventional, GGA formulations and eliminate the requirement to take the product with food. The present invention also provides controlled release compositions of GGA which eliminate the need to take the drug three times a day. A preferred dosage form of the invention is a solid dosage form, although any pharmaceutically acceptable dosage form can be utilized.

The present invention further relates to a controlled release composition which produces a plasma profile substantially similar to the plasma profile produced by the administration of two or more IR dosage forms given sequentially. For example, a controlled release composition may, in operation, deliver GGA in a continuous manner, preferably during a period of up to twenty-four hours. Another object of the invention is to formulate the dosage in the form of a diffusion controlled formulations, or osmotic controlled formulations or transdermal applications.

Molecular chaperons are proteins which mediate protein folding. They bind non-covalently to exposed surfaces of proteins that are newly synthesized or are denatured or miss-folded, and assist them to fold into correct conformation. The term “molecular chaperon,” as used herein, refers to protein which assists other proteins to fold into correct or active conformations, usually by non-covalently binding to the proteins. Chaperons not only assist in the correction and restoration of proteins newly synthesized but also of those that have been denatured or miss-folded. Molecular chaperons include, among others, heat shock proteins (HSP) including hsp70 and hsp72.

In recent years, there have beets reports that the mechanism of action of geranyl-geranyl acetone (GGA) is mediated through the induction and expression of identification of specific genetic polymorphisms related to heat shock protein, and/or commonly miss-folded proteins. Impaired heat shock protein mediated degradation of abnormal protein aggregates in dementia can thus be identified as described herein and treated by a novel agent targeted to correcting this altered pathway. The genes targeted specifically by this therapy belong to a family of heat shock proteins, including heat shock protein 70, which acts as a chaperone to reduce abnormal protein accumulation in the CNS.

As mentioned, individuals identified by single nucleotide polymorphisms related to dementia risk, or identified by other state of the art means which demonstrate abnormal protein accumulation in the brain, such as tagged protein radiotracer studies, are hereby disclosed as being candidates for the administration of an agent which restores impaired protein degradation, such as GGA, based upon its ability to up regulate heat shock proteins. GGA may decrease or eliminate the risk of developing dementia and/or inhibit its progression, via GGA's promotion of the degradation of abnormal proteins. GGA is herein proposed as a heat shock protein-inducing agent, may have therapeutic application to individuals at risk of dementia who exhibit polymorphisms in clusterin, which is a heat shock protein vital to amyloid and hyperphosphorylated tau degradation.

In a cell model, GGA increased the levels of Hsp70, Hsp90, and Hsp 105 and inhibited cell death. Oral administration of GGA also up-regulated the expression of HSPs in the central nervous system, and promotes the expression of HSP70 in the brain. Thus, GGA may be used as a neuroprotective agent in individuals with abnormal clusterin function and reduced ability to mediate protein degradation. Geranylgeranylacetone (GGA), a potent HSP inducer, reduces amyloid oligomer levels and aggregates. Neurotoxicity may also be reduced by HSP induction via the administration of geranylgeranylacetone (GGA), which can directly interrupt amyloid oligomer formation and tau accumulation.

Small HSPs which are induced by GGA can directly interrupt amyloid oligomer formation, and the in vivo protective effects of the small HSPs induced by GGA administration on the development of abnormal protein aggregation associated with neurodegenerative diseases has been previously undisclosed as a therapeutic use of the compound for individuals displaying polymorphisms in the clusterin gene. Oral administration of GGA not only results in up-regulation of the expression level of HSP70, but also reduces amyloid oligomer levels and aggregates.

GGA, as a heat shock protein inducer, may correct impaired protein degradation associated with individuals at risk of developing or who already have symptoms of dementia. The administration of GGA may allow the up-regulation of degradation processes associated with the ubiquination mediated degradation to facilitate amyloid degradation. In other words, one of the primary processes associated with Alzheimer's is the inability to degrade amyloid fibrils and tau, as a result of impaired protein ubiquination degradation processes. By identifying individuals at risk for dementia, ideally presymptomatic or in early stages of the disease, the administration of GGA may correct the degradation process by up-regulation of key heat shock proteins which are associated with the protein ubiquination process.

Other drugs that act to up-regulate heat shock proteins may be used as HSP enhancers, including, for example BRX-220 (a co-inducer of HSPs), certain proteasome inhibitors (e.g., MG132), Cyclosporine A, cyclopentenone prostaglandins, Valproic acid, HDAC inhibitors, antibiotics of the tetracycline family including tetracycline, minocycline and geldamycin, angiotensin receptor inhibitors, dihydropyridines, phosphodiesterase inhibitors (particularly those which are CNS selective including PD4a and PD4B and PD10), and certain atypical neuroleptics such as clozapine, and the like. Drugs (such as GGA) which up-regulate HSP-70 may be of particular interest.

### Glial Modulation

Tianeptine is one example of a drug that may be used as described herein to moderate glial cell activity. Tianeptine by exerting effects on GLT-1 activity or expression. Tianeptine has been generally described for the treatment of neurological and neurodegenerative disorders. For example, PCT/FR00/00865 filed Apr. 6, 2000 relates to the use of Tianeptine, of isomers thereof and of salts thereof, in obtaining medicaments intended for the treatment of neurodegenerative pathologies. French Patent Specification FR 2 635 461 describes the use of Tianeptine and compounds thereof in the treatment of stress, and FR 2 716 623 describes the use of the (+) isomer of Tianeptine in obtaining medicaments intended for mnemo-cognitive disorders. However, these patents do not instruct a clinician or researcher on which patients are best suited to receive Tianeptine, especially if overt symptoms of a neurodegenerative disease have yet to become clinically apparent. Further, the prior art does not describe or suggest the use of Tianeptine specifically as a prophylactic agent, where the administration of said treatment is more likely to be efficacious. We herein propose a link between Tianeptine and GLT-1 activity and/or expression in a way that may protect against the neurodegeneration leading to dementia, being of particular relevance to expression of APOE4 and or APOJ.

Expression of GLT-1 (the principle glutamate transporter) is reduced in astrocytes of Alzheimer's dementia (AD) cases with APOE epsiloh4-associated subtype of AD, suggesting that new means to assess GLT-1 impairments and apply specific remedies to restore its function may represent a novel and vastly improved method in treating neurodegenerative disorders, which for the first time teaches methods which inform the clinician which patients are most appropriate to consider the use of Tianeptine. In addition (or alternative) to Tianeptine, other drugs known to modulate GLT-1 (e.g., activity and/or expression) may be used. Other agents may also be used to modulate (LT-1 activity or expression. For example, scyllo-inositol is a stereoisomer of inositol, also known as scyllital, cocositol, quercinital, and 1,3,5/2,4,6-hexahydroxycyclohexane, that may enhance glial activity. Scyllo inositol is a naturally occurring plant sugar alcohol found most abundantly in the coconut palm. Scyllo-Inositol may up-regulate the activity of glia, including activity of GLT-1, possibly because it provides a source of energy to glial cells, resulting in an increase in GLT-1 activity.

As described in greater detail below, any of the drugs or agents described herein may be used alone or in combination,

### Magnetic Resonance Spectroscopy

As mentioned above, without the activity of GLT-1/EAAT2, glutamate may build up and kill cells in a process called excitotoxicity, in which excessive amounts of glutamate acts as a toxin to neurons by triggering a number of biochemical cascades. The GLT-1/EAAT2 also allows glutamate to be recycled for repeated release. GLT-1 derangements can be measured in vivo by magnetic resonance spectroscopy (MRS), and specifically ¹³C MRS. Under normal physiological conditions, the oxidation of glucose through the TCA cycle is the primary source of energy for the brain. ¹³C label isotoptically labeled precursors move through metabolic cycles in different cellular compartments. ¹³C-MRS has been successfully used to provide time-resolved observations of label incorporation into the carbon backbones of Glu, Gln, and GABA. [2-13C] acetate results in selective uptake by glial cells, thereby allowing a direct measurement of the rate of glial metabolism. The subsequent flow of the isotopic-label from glial-produced Gln into neuronal GABA and Glu potentially allows for separate measurements of the GABA/Gln and Glu/Gln cycles. The ability of ¹³C-MRS to selectively measure the flux of Glu through the glial compartment under different experimental conditions allows for measuring the pharmacological modulation of glial targets to be directly tested in clinical studies of Alzheimer's disease.

For example, MRS may be used to detect a problem with glutamate clearance (and, indirectly, a problem with clearance of amyloid in some individuals) and/or to monitor the use of any of the therapies described herein. For example, MRS may be used to monitor the use of Tianeptine; Tianeptine may be prescribed as indicated herein to affect GLT-1 expression/activity in glia, and therefore help to restore its protein malfolded chaperone activity. On a molecular level, reduced expression of glial enzymes, reduced glial densities and impaired expression of enzymes required for amyloid removal.

Because pre-clinical dementia is not associated with gross tissue pathology, the availability of research tools such as MRS to assess the brain is critical to elucidating the mechanisms of pathology, obtain an accurate diagnoses and ability to monitor therapeutic interventions.

MRS may be a useful technique and biomarker to include in preventive trials in presymptomatic or early stage individuals and provide guidance on inclusion clinical trials related to the use of Tianeptine or GGA for dementia. MRS is ideally suited for early diagnosis and differential diagnosis of AD, a role in prognosis of disease severity, a role in predicting future progression to AD in patients with mild cognitive impairment and tracking disease progression. However, current modalities have limitations which the current disclosure addresses in order to maximize the potential of this technology to objectively assess the clinical effects of a disease-modifying treatment in Alzheimer's disease.

On a molecular level, reduced expression of glial function is a fundamental mechanism in Alzheimer's diseases. This disturbance appears to be potentiated by selective genotypes which impair normal glial cell function. Excitatory amino acid transporters (e.g., GLT-1) are embedded in glial processes which act as the primary means of clearing extrasynaptic glutamate. Reduced glial densities and impaired expression of GILT-1 may be an important biomarker in Alzheimer's, as previously mentioned.

Given that up to 90% of extracellular Glu clearance is mediated by astrocytes through the activity of GLT1, methods which can quantify the activity of GLT-1 can be utilized to monitor the course of disease in Alzheimer's as well as be used in clinical trials for drugs which act upon this protein.

Magnetic resonance spectroscopy (MRS) defines neurochemistry on a regional anatomical basis by acquiring a radio-frequency signal with chemical shift from one or many voxels on MRI. For example, neurometabolites of interest may be localized on a horizontal scale (chemical shift), and their relative metabolite concentrations are determined from the metabolite's peak height decline.

In proton magnetic resonance spectroscopy (¹H-MRS), the signal intensity is proportional to metabolite concentration but it is also affected by a large number of variables which reduce the signal to noise ratio. These include spectral resolution limitations, acquisition time, eddy current artifacts, data processing, incomplete water suppression, and overlap of metabolite peaks. It has also been shown that differences in data processing are a dominant source of inter-study variability; for example, LCModel and AMARES, two widely used commercially available spectroscopy toolkits, may perform differently in terms of sensitivity to noise, linewidth and baseline.

It has proven difficult for MRS to obtain reproducible measurements from the temporal lobe, a primary area of gross pathology in dementia. The voxel size used (8 cm³) in MRS to obtain the sufficient signal-to-noise ratio (SNR) is larger than the volume of the temporal lobe, causing partial volume averaging of the surrounding tissue and decreasing the anatomic specificity of the measurements. The increased availability of scanners with 3 or 4Tesla magnetic fields can improve the sensitivity of MRS studies but also yields higher field distortions as the signal increases.

As a consequence of the inherent difficulties in obtaining reliable absolute concentrations of neurometabolites of interest in dementia, in-vivo MR spectroscopy has had limited widespread adoption of the technology clinically. It is therefore critical in clinical practice to select an established protocol of parameters to ensure uniformity. Acquisition variables which need standardization and internal consistency include metabolite relaxation rates, pulse sequence parameters, voxels of interest, artifact suppression techniques and radio-frequency coil sensitivity. Systems and methods for determining exemplary values for acquisition parameters (such as repetition times, flip angles, echo times, receiver bandwidths, number of averages), with the exemplary values being chosen to increase the signal-to-noise ratio in the metrics of interest as they relate to improving the sensitivity and specificity of brain degenerative changes in dementia are required.

The chemicals quantifiable with MRS include N-Acetyl-Aspartate (NAA), myoinositol (mI), and glutamate. Acquisition parameters having the exemplary values may be used for the given imaging time to image N acetylaspartate, myoinositol and glutamate accurately and unequivocally.

The term “GLX” (Glx) is normally used to designate the single peak containing the amino acid neurotransmitters Glutamate (Glu). Gamma-Aminobutyric Acid (GABA), and Glutamine (Gln) as ¹H-MRS signals from Glu and Gln are complicated by the interaction of neighboring protons. Brain in vivo concentrations of Glu are approximately 8-13 times that of GABA, and the ratio of Glu/Gln ranges from 2.4-3.8; therefore, alterations in Glx are typically attributed to altered Glu concentrations, but may in fact be measuring other metabolites as well. This has clinical implications in trials designed to specifically and accurately measure glutamate, a primary metabolite of interest in dementia.

MRS metabolite abnormalities in dementia to date have been characterized by an elevated myoinositol (mI) and decreased NAA, which may be present several years before the onset of symptoms, suggesting the utility of the MRS in presymptomatic disease detection. NAA is the most prominent H-MRS peak and is a marker of neuronal function. Reduction in NAA levels measured by -MRS is a recognized marker of neuronal loss in several psychiatric and neurological disorders. For example, U.S. Pat. No. 5,617,861 claims the use of N acetylaspartate through MRS as a diagnostic biomarker for dementia.

Baseline mI levels are higher in patients with mild cognitive impairment and Alzheimer's disease than the controls and demonstrates longitudinal elevation in the course of Alzheimer's disease. Increased Myoinositol (mI) indicates elevated neuroglial concentration, a “reactive astrogliosis” associated with the pathogenesis of Alzheimer's. Astrocyte dysfunction, as described earlier, is a primary pathological mechanism as well as a therapeutic target for the disease.

However, limited no measurement reproducibility exists due to its low signal to noise ratio (as the mI signal is split between six coupled protons), as well as to its spectral overlap with a number of other brain metabolites, including glutamate (Glu), and glutamine (Gln).

A number of acquisition strategies has been proposed to discriminate various spectra in this regard and should be regarded as a standard operating procedure to accurately assess this and other metabolites. Many of these are known to those skilled in the art, but for practical purposes have not been incorporated as a standard procedure in dementia clinical application.

Two-dimensional (2D) MRS adds a second frequency dimension to each spectrum to selectively reduce the overlapping, background resonances of myoinositol for instance.

By acquiring multiple 1D spectra with incrementally longer TEs and applying double Fourier transform on the set of spectra to produce a 2D spectrum, overlapping resonances can be more accurately discriminated. The spectroscopic image can be formed using exponential apodization in the time domain (2 Hz Lorentzian width) and no apodization in kspace.

The voxels of interest (VOI) also requires consistency and standardization. The VOI needs to be centered to ensure the same locations in each subject, voxels encompassing an 8-cm³ (2×2×2-cm) voxel, prescribed on a midsagittal T1-weighted image, included right and left posterior cingulate gyri in subjects. Point resolved spectroscopy (PRESS) pulse sequences can be applied to these voxel with repetition time/echo time=2,000/30 msec.

Water suppression can be achieved by measures known to those skilled in the art. For instance, point resolved spectroscopy sequence with global water suppression by means of is chemical shift selective saturation (CHESS) pulse. A chemical shift selective pre-excitation includes an excitation bandwidth of about 1.8 ppm to 2.5 ppm. Suppression Bandwidth is the range of frequencies in the spectrum that is effectively suppressed by a suppression technique by selective pre-excitation or band selective inversion with gradient dephasing. Exciting the neuronal tissue via slice selective spin-echo excitation and suppressing non-NAA magnetic resonance signals by a combination of band selective inversion with gradient dephasing (J resolved), produces a suppression band width which includes the water resonance at about 4.7 ppm. The suppressing step (b) can suppress magnetic resonances down field from 2.5 ppm, allowing for a more accurate discrimination of the neurometabolites of interest.

Improving metabolite quantification homogeneity can also be optimized with shimming procedures. Increasing the strength within the probe volume with higher Telsa MRI leads to broadening of spectral peaks and a reduction of the signal-to-noise ratio (SNR), but this may lead to quantification errors. Non-homogeneous distributions of the magnetic field can be made homogeneous with shimming procedures.

Shimming can be achieved by placement of configurations of ferromagnetic objects with proper size and positioning into the magnetic field in order to improve the field homogeneity within the sensitive probe volume. Active shimming was developed to provide highly accurate field cancellation with a flexible interface. Magnetic fields can be fully described by spherical harmonic functions. With a set of appropriate electomagnetic coils, each generating a magnetic field component that corresponds to one spherical harmonic, the field inhomogeneity can be minimized by superposition of a shim field of the same magnitude but opposite sign to the distortion. Conventional active shimming has a restriction in high field strength applications. However, there is a continuous trend towards the use of higher magnetic fields for both research and clinical scanners, since the signal to noise ratio improves at least linearly in MR imaging and spectroscopy with increased magnetic field. Furthermore, higher fields lead to better spectral dispersion in MR spectroscopy, which is advantageous particularly for the separation of glutamate (Glu) from glutamine (Gln).

However, the benefit of better signal to noise and simplified spectra at high field can only be exploited if optimal shimming is achieved, as field distortions due to susceptibility effects also increase at higher field strength. Increased field distortions require stronger shim fields, potentially exceeding the capabilities of the conventional active shim devices. In addition, regions of strongly differing magnetic susceptibilities like tissue-air transitions, e.g. in the vicinity of the cranial bone, require shim fields beyond those provided by active shimming.

Decomposing the field inhomogeneities into first and second order spherical harmonic functions, determining primary shim terms derived from the second order spherical harmonic functions, wherein the primary shim terms yield a passive shim field adapted to a targeted shim field, scaling optimized shim terms for increasing a similarity of the passive shim field with the targeted shim field, and constructing the modular shin sheets on the basis of the optimized shim terms. While this procedure has been previously disclosed, its specific clinical application in dementia testing and assessment of drug trials has not been previously disclosed.

In vivo ¹³C spectroscopy provides a unique way to study metabolic fluxes of glutamate in human brain and can also be applied to assess early disease detection, disease progression and the effects of Tianeptine, other GLT-1 modulating agents or other potential meuroprotective agents in Alzheimer's disease. Radiolabeled MRS may be improved through hyperpolarization techniques. In hyperpolarization techniques, a sample of a labeled imaging agent, for example ¹³C Pyruvate or another similar polarized metabolic imaging agent, is introduced or injected into the subject being imaged.

Glutamate 13C- can be enriched by intravenous [2,5-13C]glucose infusion for selective 13C-enrichment of the glutamate pool. 13C-enrichment can be followed by [12C]glucose infusion to displace 13C from the small glial GLU pool, and the observed rate of displacement represents a reasonable estimate for the rate of glial uptake and GLT-1 function.

C5 glutamate signal enhancement can also be achieved via a low-power nuclear Overhauser effect (NOE). The nuclear Overhauser effect (NOE) is a cross-relaxation phenomenon which involves two magnetically active nuclei in close proximity. Signal enhancement via the NOE mechanism can be realized using a low proton pulse power. NOE together with a proton decoupling have been shown to significantly enhance the signal-to-noise ratio and resolution in ¹³C MRS as the carbonyl carbon (C5) of glutamate is subject to dipolar interaction with surrounding water protons.

While ¹³C MRS techniques and NOE have been ‘previous’ disclosed, its application to the use of pre-dementia testing and as an assessment of GLT-1 modifying agents has not been previously disclosed.

### EXAMPLES

In use, the methods and systems described herein may include a procedure for first determining a subject's risk for developing dementia, and then prescribing and/or administering a composition (including one or more drugs) to prophylactically treat at-risk patients. The compositions may include one or more agents to enhance amyloid clearance (e.g., to increase HSP expression/activity) and/or one or more agents to enhance amyloid clearance (e.g., to modulate GLT-1 expression/activity).

The procedure for determining if a patient is at risk (or has an elevated level of risk) may include determining the patient's genetic risk factors, including the presence of one or more (or a combination of) genetic markers including single nucleotide polymorphisms (SNPs), particularly in the APOE and/or Clusterin genes. In addition or alternatively, the step of determining if a patient is at risk may include determining if the patient has an increase in amyloid plaque. For example, amyloid build-up may be detected visually (and possibly non-invasively) by examining blood vessels, including those in the patient's eye (e.g., retina). A marker may be used to detect amyloid, such as the compound Florbetaben, which binds directly to beta-amyloid, and can be used in PET molecular imaging to visualize the protein directly during or prior to the development of clinical dementia.

In some variations, the procedure for determining if a patient is at risk may include determining if the patient is at risk for early-onset/familial Alzheimer's including looking for genetic markers for early-onset Alzheimer's (e.g., mutations of PSEN1, PSEN2, APP). Such patients may also benefit from the therapies described herein.

In some variations, if the risk of developing a neurodegenerative disorder is indicated by the presence of the APOE4 and/or polymorphisms in APOJ, and/or if there is an increase in abnormal (or excessive) protein aggregation, or other enhanced genetic risk, then the subject may be prescribed and/or administered a compound or composition that induces or increases the expression of a heat shock protein, and/or modulates the expression/activity of the GLT-1 transporter. For example, the compound may be an acyclic polyisoprenoid such as geranylgeranylactone (GGA), and/or Tianeptine. The compound may be used to delay the onset, prevent and/or ameliorate a neurodegenerative disorder.

The therapy provided herein may be a specific medical prescription or treatment regime designed to modify or ameliorate the biochemical disturbance associated with the aggregation of amyloid in the CNS due to polymorphisms in the clusterin gene and certain isoforms of APOE4.

Such treatments may include the prescription and/or application of a composition consisting of an acylic polyisoprenoid such as geranylgeranylacetate (“GGA”), or any other compound or composition that can induce or increase expression of a heat shock protein. Alternatively, or additional, other agents linked to up-regulation of heat shock protein, and HSP 70 in particular, may be used. These may include: Valproic acid, HDAC inhibitors, antibiotics of the tetracycline family including tetracycline, minocycline and geldamycin, angiotensin receptor inhibitors, dihydropyridines, phosphodiesterase inhibitors (particularly those which are CNS selective including PD4a and PD4B and PD10), and certain atypical neuroleptics such as clozapine.

The molecule, compound or composition applied or offered may modify the genes associated with protein aggregation by up-regulating the heat-shock proteins and thereby correcting the genetic defect which impairs normal protein aggregation inhibition.

In some variations, the therapeutic provided and/or prescribed (e.g., a molecule, compound, composition, etc.) can be formulated in an extended release formulation to improve compliance in patients with cognitive disorders.

For example, described herein are compositions for treating Alzheimer's dementia, the composition comprising a mixture of compounds for enhancing HSPs/amyloid clearance and compounds for modulating GLT-1 activity in glial cells. Any appropriate compounding may be used. In particular, the composition may be formulated as a delayed-release composition. For example, described herein are compositions for treating Alzheimer's dementia, the composition comprising a mixture of geranylgeranylacetone (GGA) and Tianeptine.

### Example

In one example, an individual visits his or her health care worker because of a concern related to risk of developing Alzheimer's disease. The subject may be asymptomatic (or preclinical) for Alzheimer's or dementia. Alternatively, the person may be symptomatic, displaying signs of cognitive dysfunction associated with dementia or depressive symptoms. In either case, the health care worker may obtain a sample of genetic material which is analyzed for the presence of polymorphisms in the clusterin and APOe gene. SNPs at the CLU (also known as APOJ) gene (e.g., rs11136000) have been associated with dementia. In some variations, the patient may be subjected to a diagnostic radiographic study such as described earlier which is able to detect abnormal protein in the brain, and/or abnormal activity of GLT-1.

If an increased risk is determined, the subject may be prescribed and/or treated with a specific therapeutic compound as described herein. For example, in some individuals, APOE4 polymorphisms will be detected; in such subjects, Tianeptine may be prescribed with the intended goal to delay, retard or inhibit progression of said disorder. The Tianeptine may be prescribed in conjunction with (or co-compounded with) another compound which induces HSP, such as GGA. Compositions including both an up-regulator of a HSP and/or modulator of GLT-1 may be administered and may provide enhanced protection over either the up-regulator of HSP or glial based GLT-1 function. In some variations a compositions may include multiple agents that up-regulate HSPs and/or multiple agents that regulate GLT-1.

Before symptomatic memory toss, healthy apolipoprotein E epsilon4 (APOE epsilon4) and clusterin gene polymorphism carriers may demonstrate accelerated longitudinal decline on memory tests, suggesting the existence of a transitional state between normal aging and mild cognitive impairment (MCI). For example, APOE epsiton4 homozygotes have higher rates of cognitive domain decline than APOE epsilon4 heterozygotes or non-carriers before the diagnosis of MCI and AD, thus confirming and characterizing the existence of a pre-MCI state in this genetic subset. Thus, an overriding principal and motivation for the current discovery is the critical importance of early detection of individuals in pre symptomatic or early symptomatic stages of dementia. This detection can be achieved by gene testing and/or brain imaging as discussed. However, what is vitally missing from the field is not only a means of diagnosis, hut also a remedy which addresses the identified individual.

Neuroprotective strategies based upon the modulation of heat shock proteins and/or regulation of GLT-1 based glial activity are a previously undisclosed aspect of this invention. The implementation of said therapy represents a profound advancement to the field of dementia treatments, which are currently palliative only. currently, there are no disease-modifying treatments which address the primary neuropathological abnormalities associated with dementia. The application of said therapy, based upon early detection, provides a previously undisclosed means to inhibit, prevent or retard the onset of dementia.

Additional details pertinent to the present invention, including materials and techniques, may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” only and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the examples described herein, but only by the plain meaning of the claim terms employed.

What is claimed is:

1. A method of prophylactically treating Alzheimer's dementia, the method comprising providing one or both of Tianeptine and GGA to a patient having one or both an APOE4 polymorphism and a polymorphism in APOJ.

2. A method of prophylactically treating Alzheimer's dementia, comprising providing Tianeptine to a patient having the APOE4 polymorphism.

3. The method of claim 1 or 2, further comprising: determining that the patient has the APOE4 polymorphism.

4. The method of claim 1 or 2, further comprising: determining that the patient has the APOE4 polymorphism and a polymorphism in APOJ.

5. The method of claim 1 or 2, further comprising providing both Tianeptine and GGA.

6. A method of prophylactically treating Alzheimer's dementia, comprising providing Tianeptine to a patient having one or both the APOE4 polymorphism and a polymorphism in APOJ.

7. The method of claim 6, further comprising providing both Tianeptine and GGA.

8. A method of prophylactically treating Alzheimer's dementia, comprising providing both Tianeptine and GGA to a patient having one or both the APOE4 polymorphism and a polymorphism in APOJ.

9. A method of prophylactically treating Alzheimer's dementia, the method comprising determining if the patient has a polymorphism in APOE; and providing Tianeptine to the patient having the APOE4 polymorphism.

10. A method of prophylactically treating Alzheimer's dementia, the method comprising determining if the patient has a polymorphism in APOE and APOJ; and providing Tianeptine to the patient having either or both the APOE4 polymorphism and an APOJ polymorphism.

11. A method of prophylactically treating Alzheimer's dementia, the method comprising determining if the patient has a polymorphism in one or both of APOE and APOJ; and providing one or both of a glial modulator and a HSP enhancer.

12. A test for determining if a patient will benefit from the prophylactic treatment for Alzheimer's dementia with one or both of a glial modulator and a HSP enhancer, the method comprising: indicating if the patient will benefit from treatment with one or both of an enhancer of glial function and a HSP enhancer based on the presence of one or both of an APOE4 polymorphism and a polymorphism in APOJ.

13. The test of claim 12, further comprising determining if the patient has one or both the APOE4 polymorphism and a polymorphism in APOJ.

14. A test for determining if a patient will benefit from the prophylactic treatment for Alzheimer's dementia with one or both of an enhancer of glial function and a HSP enhancer, the method comprising: indicating if the patient will benefit from treatment with one or both of Tianeptine and GGA based on the presence of one or both of an APOE4 polymorphism and a polymorphism in APOJ.

15. A composition for the prophylactic treatment of Alzheimer's dementia, the composition comprising Tianeptine, and an HSP enhancer.

16. The composition of claim 15, wherein the HSP enhancer is GGA.

17. The composition of claim 15, wherein the HSP enhancer may be chosen from a list consisting of: Valproic acid, HDAC inhibitors, antibiotics of the tetracycline class, BRX-220, MG132, Cyclosporine A, cyclopentenone prostaglandins, angiotensin receptor inhibitors, dihydropyridines, phosphodiesterase inhibitors, atypical neuroleptics, and GGA.

18. A method to determine the effects of prophylactic Alzheimer's therapy in a clinical trial or in practice through the employment of magnetic resonance spectroscopy (MRS), the method comprising: monitoring the brain of a patient receiving one or both of a modulator of glial function and a HSP enhancer with MRS.

19. The method of claim 18, wherein monitoring the brain comprises monitoring glutamate labeled glial based function.

20. The method of claim 18, further comprising prophylactically treating the patient with one or both of a modulator of glial function and a HSP enhancer with MRS.

21. The method of claim 18, wherein a signal to noise ratio of the MRS is increased by various means to accurately assess N acetylaspartate, myoinositol and glutamate.

22. The method of claim 18, wherein monitoring comprises monitoring the brain of a patient receiving one or both of tianeptine and GGA.

23. The method of claim 18, where the monitoring comprises monitoring the effects of said therapy on GLT-1 function.

24. The method of claim 18, wherein monitoring comprises monitoring one or more of: n acetylasparate, inositol and glial uptake of glutamate.

25. The method of claim 18, further comprising applying shimming procedure to enhance the MRS.

26. A method assessing risk of cognitive decline by estimating amyloid burden, the method comprising:
determining a subject's genotype for APOE and APOJ;
determining the subject's amyloid burden from the subject's APOE and APOJ genotype;
estimating a risk of cognitive decline based on the amyloid burden.

27. The method of claim 26, further wherein estimating the risk of cognitive decline comprises classifying the genetic risk on a scale including high, moderate and low.

28. A method of determining if a patient will respond to anti-inflammatory, or conversely, a neuroimmunomodulatory therapy to treat or prevent dementia, the method comprising:
determining a subject's genotype for APOE and APOJ;
indicating that treatment with anti-inflammatory agents is contraindicated in patient's having an APOE4 polymorphism and/or a polymorphism in APOJ.

29. A method of prophylactically treating Alzheimer's dementia by determining the patient's propensity for amyloid accumulation and inability to clear amyloid, the method comprising:
determining if the patient has an impaired or reduced ability to degrade amyloid by MMP-9 activity based on the patient's APOE genotype; and
determining if the patient's chaperone/HSP activity is impaired with subsequently reduced amyloid degradation based on the patient's APOJ genotype.
